(19) United States
(12) Patent Application Publication
(10) Pub. No.: US 2026/0356776 A1
(43) Pub. Date: Nov. 12, 2026

(54) METHOD FOR PRODUCING OLEFINS THROUGH DEHYDROGENATION-CRACKING REACTION OF SATURATED HYDROCARBONS

(71) Applicant: KOREA RESEARCH INSTITUTE OF CHEMICAL TECHNOLOGY, Daejeon (KR)

(72) Inventors: Dae Sung PARK, Daejeon (KR); Yong Ki PARK, Daejeon (KR); Do Kyoung KIM, Daejeon (KR); Ha Won PARK, Daejeon (KR); Won Choon CHOI, Daejeon (KR)

(73) Assignee: KOREA RESEARCH INSTITUTE OF CHEMICAL TECHNOLOGY, Daejeon (KR)

(21) Appl. No.: 19/122,554

(22) PCT Filed: Oct. 18, 2023
(86) PCT No.: PCT/KR2023/016097

§ 371 Date: Apr. 18, 2025

(30) Foreign Application Priority Data
Oct. 19, 2022 (KR) 10-2022-0134555

(51) Int. Cl. C07C 11/08; C07C 4/06; C07C 5/333; C07C 11/06

(52) U.S. Cl. CPC C07C 11/08; C07C 4/06; C07C 5/3337; C07C 11/06; C07C 2521/02; C07C 2529/06; C07C 2529/85

(57) ABSTRACT

The present invention relates to a method for producing olefins through the dual dehydrogenation-cracking reaction of paraffins. The method for producing olefins according to the present invention efficiently catalytically decomposes paraffins with a low number of hydrocarbon atoms to increase the yield of olefins, while simultaneously reducing the production of methane, whereby olefins can be produced with excellent efficiency at lower temperatures.

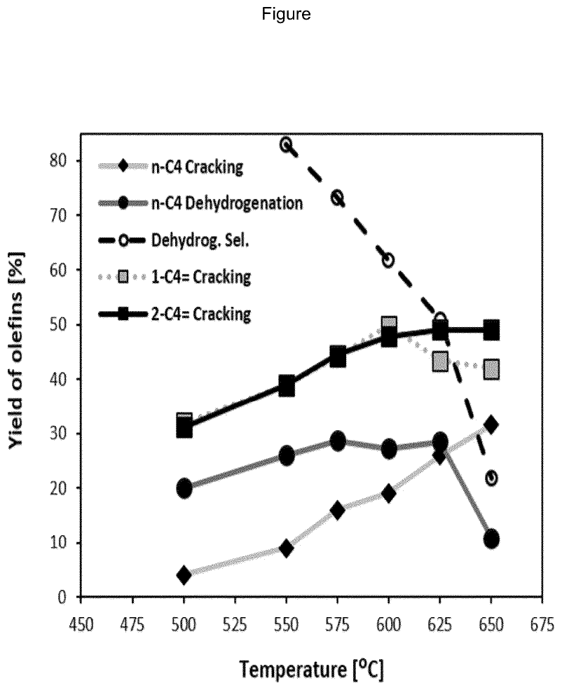

### CROSS REFERENCE TO RELATED APPLICATIONS

This application is a National Stage of International Application No. PCT/KR2023/016097 filed Oct. 18, 2023, claiming priority based on Korean Patent Application No. 10-2022-0134555 filed Oct. 19, 2022, the entire disclosures of which are incorporated herein by reference.

### TECHNICAL FIELD

The present invention relates to a method for producing olefins through dehydrogenation-cracking reaction of saturated hydrocarbons.

### BACKGROUND ART

The process of producing light olefins such as ethylene or propylene through reaction of saturated hydrocarbons is the most important process in the petrochemical industry and has the largest market. In particular, a thermal decomposition process using naphtha feedstock is a main process of producing light olefins and is most widely used in the petrochemical industry.

There are two major recent trends in a process of producing light olefins through a cracking reaction. First, carbon emissions are reduced by replacing the energy-intensive thermal decomposition process which is operated at 900 to 1,000 °C with a catalytic decomposition process and using a catalyst at a low temperature of 750 °C or lower. Second, light olefins are produced without additionally increased crude oil production by replacing naphtha feedstock with an unused or recycled oil fraction. In particular, in order to reduce the risk of oil price fluctuations and secure cost competitiveness, a study of a process using LPG as a raw material thermal decomposition process of naphtha is widely in progress. In addition, since C3-C4 LPG having fewer hydrocarbons than naphtha has a lower carbon dioxide emission factor than naphtha, it is also favorable in terms of carbon emissions. However, since LPG proceeds the thermal decomposition at around 850 °C, and uses basic chemical fuels such as olefins or BTX along with naphtha feedstock as production fuels, energy usage may be further reduced by lowing an operation temperature and optimizing the process through catalytic decomposition of LPG. In addition, since paraffins have a low catalytic decomposition ability than olefins, and the catalytic decomposition activity decreases further as the number of hydrocarbons decreases, development of a new catalyst is needed to efficiently performing a catalytic decomposition reaction of a raw material only formed of paraffins having a small number of hydrocarbons, such as LPG.

Thus, a study for improving the catalytic decomposition properties of paraffin-based hydrocarbons having a small number of hydrocarbons, such as LPG, is still needed.

### DISCLOSURE

### Technical Problem

An object of the present invention is to provide a method for improving a catalytic decomposition ability of paraffins which have a small number of hydrocarbons and a lower catalytic decomposition ability than olefins.

Another object of the present invention is to provide a method for improving process efficiency and yield for producing olefins by reducing an amount of methane produced.

### Technical Solution

In one general aspect, a method for producing olefins from saturated hydrocarbons includes: bringing a saturated hydrocarbon feedstock into contact with a mixture of a dehydrogenation catalyst and a cracking catalyst in a circulating fluidized bed reactor.

In the method for producing olefins from saturated hydrocarbons of the present invention, the saturated hydrocarbon may be a C3 to C10 saturated hydrocarbon.

In the method for producing olefins from saturated hydrocarbons of the present invention, a weight ratio between the dehydrogenation catalyst and a cracking catalyst may be 2:1 to 5:1.

In the method for producing olefins from saturated hydrocarbons of the present invention, the bringing of a saturated hydrocarbon feedstock into contact with a mixture of a dehydrogenation catalyst and a cracking catalyst may be performed at 300 to 800 °C.

In the method for producing olefins from saturated hydrocarbons of the present invention, a dehydrogenation reaction with the dehydrogenation catalyst and an olefin decomposition reaction with the cracking catalyst may be performed in a single reactor.

In the method for producing olefins from saturated hydrocarbons of the present invention, the dehydrogenation catalyst may be a metal active component including cobalt and platinum supported on an alumina carrier including boron.

In the method for producing olefins from saturated hydrocarbons of the present invention, the boron may be supported at 0.5 to 2 wt % with respect to the weight of the alumina carrier, the cobalt may be supported at 1 to 5 wt % with respect to the weight of the alumina carrier, and the platinum may be supported at 0.001 to 0.05 wt % with respect to the weight of the alumina carrier.

In the method for producing olefins from saturated hydrocarbons of the present invention, the cracking catalyst may be selected from the group consisting of a SAPO molecular sieve, a ZSM molecular sieve, an MCM molecular sieve, and a combination thereof.

In the method for producing olefins from saturated hydrocarbons of the present invention, the olefin may be propylene and butene.

In another general aspect, the olefins produced by the method for producing olefins from saturated hydrocarbons are provided.

### Advantageous Effects

The method for producing olefins from saturated hydrocarbons according to the present invention increases the yield of olefins by efficiently catalytic decomposing paraffins having a small number of hydrocarbons, and may simultaneously produce olefins with excellent efficiency even at a low temperature by decreasing an amount of methane produced.

### DESCRIPTION OF DRAWINGS

The FIGURE is a graph showing the yield of olefins from n-butene, 1-butene, and 2-butene depending on temperatures.

### BEST MODE

Hereinafter, a method for producing olefins from saturated hydrocarbons of the present invention will be described in detail with reference to the accompanying drawings.

The drawings to be provided below are provided by way of example so that the spirit of the present disclosure may be sufficiently transferred to a person skilled in the art. Therefore, the present invention is not limited to the drawings provided below but may be embodied in many different forms, and the drawings suggested below may be exaggerated in order to clarify the spirit of the present invention.

Technical terms and scientific terms used herein have the general meaning understood by those skilled in the art to which the present invention pertains, unless otherwise defined, and the description for the known function and configuration which may unnecessarily obscure the gist of the present invention will be omitted in the following description and the accompanying drawings.

In addition, singular form used in the specification and claims appended thereto may be intended to include a plural form also, unless otherwise indicated in the context.

In the present specification and the appended claims, the terms such as “first” and “second” are not used in a limited meaning but are used for the purpose of distinguishing one constituent element from other constituent elements.

In the present specification and the appended claims, the terms such “comprise” or “have” mean that there is a characteristic or a constituent element described in the specification, and as long as it is not particularly limited, a possibility of adding one or more other characteristics or constituent elements is not excluded in advance.

In the present specification and the appended claims, when a portion such as a membrane (layer), an area, and a constituent element is present on another portion, not only a case in which the portion is in contact with and directly on another portion but also a case in which other membranes (layers), other areas, other constitutional elements, and the like are interposed between the portions is included.

The method for producing olefins from saturated hydrocarbons according to the present invention is characterized by including: bringing a saturated hydrocarbon feedstock into contact with a mixture of a dehydrogenation catalyst and a cracking catalyst in a circulating fluidized bed reactor.

In general, an olefin, such as ethylene and propylene, is obtained in a thermal decomposition process of naphtha. However, since a larger amount of olefin is required in the petrochemical industry, the olefin is produced also through a dehydrogenation process of a saturated hydrocarbon using a catalyst. However, a conventional dehydrogenation process mostly uses a fixed bed reactor and a moving bed reactor, but though a process using a fixed bed reactor has a high yield of olefins at the beginning of the reaction of bringing the saturated hydrocarbon into contact with the catalyst, it has a problem in that the conversion rate of saturated hydrocarbon and the yield of olefins decrease overall and a lot of energy is consumed in a regeneration process, due to deactivation of a catalyst and excessive coke production over time.

In addition, when olefins are produced by performing two steps of dehydrogenation and cracking by separating the dehydrogenation step and the cracking step, the process is complicated because the two steps of the dehydrogenation reaction and the cracking reaction are separately performed, and it has a problem that energy is wasted because cooling and heating steps should be included between the dehydrogenation reaction and the cracking reaction.

However, since the olefin is produced by performing the process including bringing a saturated hydrocarbon feedstock into contact with a mixture of a dehydrogenation catalyst and a cracking catalyst in a circulating fluidized bed reactor, according to the present invention, an efficient process may be carried out through a process in which the saturated hydrocarbon and the catalyst are injected together into a fluidized bed reactor at a very high rate to perform the reaction, and then the catalyst enters a regeneration part and a product enters a separation part. In addition, since the dehydrogenation reaction and the cracking reaction proceed simultaneously, less energy is wasted, and the process is relatively simple.

In a specific example, the saturated hydrocarbon feedstock may be a C3 to C10 saturated hydrocarbon, preferably a C3 to C6 hydrocarbon, and more preferably LPG. Generally, it is difficult to perform the dehydrogenation reaction and the cracking reaction through catalytic decomposition in a saturated hydrocarbon rather than in an unsaturated hydrocarbon, and in the saturated hydrocarbon, it is more difficult when the number of carbons is smaller. Therefore, conventionally the dehydrogenation reaction and the cracking reaction through catalytic decomposition use a hydrocarbon having 10 or more carbon atoms as a feedstock, but in the present invention, the dehydrogenation reaction and the cracking reaction through catalytic decomposition may proceed even at a low temperature, using a saturated hydrocarbon having 10 or less carbon atoms, specifically pure paraffins which may be obtained from crude oil or shale, more specifically LPG as a feedstock.

In a specific example, the dehydrogenation catalyst and the cracking catalyst may be used in a mixed form, and thus, an olefin may be produced from saturated hydrocarbons in a circulating fluidized bed reactor. Herein, a weight ratio between the dehydrogenation catalyst and the cracking catalyst may be 0.1:1 to 5:1, preferably 0.5:1 to 5:1, and more preferably 2:1 to 5:1. When the dehydrogenation catalyst and the cracking catalyst are used in the range of the weight ratio, decreased selectivity of ethylene may be prevented while maintaining the benefit of a decreased amount of methane produced and an increased amount of olefin produced.

In a specific example, the bringing of a saturated hydrocarbon feedstock into contact with a mixture of a dehydrogenation catalyst and a cracking catalyst may be performed at 300 to 800 °C, preferably 500 to 700 °C, and more preferably 575 to 600 °C. Generally, since the dehydrogenation reaction and the cracking reaction are equilibrium reactions, they require a high reaction temperature, and according to the method for producing olefins of the present invention, the reaction proceeds at a relatively low temperature, so that both the conversion rate and the selectivity of the saturated hydrocarbon into the olefin may be satisfied.

In a specific example, the dehydrogenation reaction with the dehydrogenation catalyst and the olefin decomposition reaction with the cracking catalyst may be performed in a single reactor. When the dehydrogenation reaction proceeds, a ratio of produced hydrogen is high, and a reaction speed may be slow due to competition between hydrocarbons and hydrogen for catalyst adsorption, and when the dehydrogenation reaction and the olefin decomposition reaction are performed in the same reactor, the problem may be prevented.

In a specific example, the dehydrogenation catalyst may include an active component in a carrier including an additional component, and preferably, may include cobalt and platinum in an alumina carrier including boron.

The carrier may be selected from the group consisting of alumina, molecular sieves, kaolin, diatomite, silica, or a combination thereof, and preferably, alumina may be selected. The alumina carrier preferably has γ to θ phases at a producing temperature of 550 to 850 °C which is equivalent to or higher than a dehydrogenation reaction temperature and may have a surface area of 80 to 300 m²/g within the range. When the carrier is produced at a temperature lower than the dehydrogenation reaction temperature, thermal deformation of the catalyst may occur during the dehydrogenation reaction, and when the carrier is produced at a temperature higher than the dehydrogenation reaction temperature, it may have a smaller catalyst surface area due to crystallization of the carrier, which may cause a problem of inhibiting mass transfer for catalytic activity when in contact with a reactant, but the dehydrogenation catalyst according to the present invention may prevent the problem.

The active component may be selected from the group consisting of cobalt, platinum, chromium, zirconium, lead, nickel, chromium oxide, gallium oxide, vanadium oxide, or a combination thereof, and preferably, may be selected from cobalt and platinum. Herein, the cobalt may be supported at the content of 1 to 10 wt %, preferably 1 to 5 wt % with respect to the weight of the alumina. When the cobalt is included within the content range, formation of crystalline oxides may be prevented and also the yield may be increased. In addition, platinum may be supported at the content of 0.001 to 0.1 wt %, preferably 0.001 to 0.05 wt % with respect to the weight of the alumina. When the platinum is included within the content range, a side reaction which produces by-products such as methane or ethane is suppressed, and the conversion rate of saturated hydrocarbon may be increased. That is, the selectivity of the unsaturated hydrocarbon compound may be increased through cobalt, and also a low conversion rate may be supplemented through platinum.

The additional component may be selected from the group consisting of tin, boron, alkali metal, alkaline earth metal, or a combination thereof, and preferably, boron may be selected. Herein, the content of boron may be 0.1 to 5 wt %, preferably 0.3 to 3 wt %, and more preferably 0.5 to 2 wt % with respect to the weight of the carrier. When an additional component is included within the content range, boron effectively suppresses a side reaction site of the carrier and suppresses coke deposition and by-product production.

In a specific example, a method for producing the dehydrogenation catalyst is characterized by including: (a) impregnating alumina with a solution including boron and firing the solution to produce a boron-alumina carrier; (b) producing a solution including a metal active component; (c) impregnating the boron-alumina carrier with the solution including the metal active component and drying; and (d) firing the boron-alumina carrier on which the metal active component is supported.

In a specific example, the boron-alumina carrier may be fired at 400 to 600 °C in (a). When it is fired at a temperature within the range, a decrease in a surface area by a change in a carrier phase may be prevented. In addition, the boron-alumina carrier on which the metal active component is supported may be fired at 700 to 900 °C in (d). When it is fired at the temperature within the range, formation of nano-sized crystal phases is prevented, so that it may be used as a dehydrogenation catalyst without causing an oxidation-reduction reaction.

In a specific example, the cracking catalyst refers to a catalyst used in a decomposition reaction of an unsaturated hydrocarbon compound, and the cracking catalyst may be a modified or unmodified molecular sieve catalyst. Herein, an appropriate molecular sieve may be a molecular sieve having a pore diameter of 4 to 7 Å, preferably, may be selected from the group consisting of a SAPO molecular sieve, a ZSM molecular sieve, an MCM molecular sieve, and a combination thereof, and more preferably, may be ZSM-5.

The olefin according to the present invention is characterized by being produced by a method for producing olefins using a process including bringing a saturated hydrocarbon feedstock into contact with a mixture of a dehydrogenation catalyst and a cracking catalyst in a circulating fluidized bed reactor.

In a specific example, the olefin may be a C2 to C4 unsaturated hydrocarbon, preferably propylene and butene. According to the method for producing olefins from saturated hydrocarbons of the present invention, an amount of methane produced may be reduced, selectivity of propylene and butene may be increased, and preferably, selectivity of propylene and butene may be 40% or more, more preferably 50% or more.

### BEST MODE FOR CARRYING OUT THE INVENTION

Hereinafter, the present invention will be described in detail by the examples. However, the examples are for describing the present invention in more detail, and the scope of the present invention is not limited to the following examples.

### <Example 1> Fluidized Bed Dehydrogenation-Cracking Catalyst Reaction

A dehydrogenation catalyst and a cracking catalyst in a mixed form were injected into a reactor to implement a dehydrogenation-cracking catalyst reaction. A cobalt-platinum/boron oxide-alumina catalyst was used as the dehydrogenation catalyst, and a ZSM-5 (Si/Al=140) catalyst was used as the cracking catalyst. A weight ratio between the dehydrogenation catalyst and the cracking catalyst was adjusted to 2:1, and the total weight of the catalyst was adjusted to 1.2 g.

### Example 2

The process was performed in the same manner as in Example 1, except that the weight ratio between the dehydrogenation catalyst and the cracking catalyst was 3:1, instead of 2:1.

### Example 3

The process was performed in the same manner as in Example 1, except that the weight ratio between the dehydrogenation catalyst and the cracking catalyst was 4:1, instead of 2:1.

### <Comparative Example 1> Cracking Catalyst Reaction

A ZSM-5 (Si/Al=140) catalyst as the cracking catalyst was injected into a reactor to implement a cracking catalyst reaction, and no dehydrogenation catalyst was used.

### <Experimental Example 1> Measurement of Temperature for Dehydrogenation-Cracking Catalyst Reaction

For implementation of a dehydrogenation-cracking catalyst reaction, C4-based compounds (n-butane, 1-butene, 2-butene) were used as a reactant and a temperature range of the dehydrogenation and cracking reactions was observed. The yield of olefins was observed after using a cobalt-platinum/boron oxide-alumina catalyst in the dehydrogenation reaction of the dehydrogenation-cracking catalyst reaction, and a ZSM-5 (Si/Al=140) catalyst in the cracking reaction, and the results are shown in the FIGURE.

Referring to the FIGURE, the yield of olefin was increased as the reaction temperature was raised in n-butane, the yield was increased up to 625 °C and then maintained later in 2-butene, and the yield was increased up to 600 °C and then was decreased in 1-butene. Next, a production yield of butenes by the dehydrogenation reaction of n-butene was shown to be the highest at 575 to 625 °C and then showed a tendency to rapidly decrease at 650 °C. That is, as the temperature increased, the conversion rate was increased, but the selectivity was decreased, and thus, it was confirmed that a temperature of 575 to 600 °C is the optimal temperature where both the conversion rate and the selectivity are satisfied.

### <Experimental Example 2> Fluidized Bed Dehydrogenation-Cracking Catalyst Reaction of n-Butane

The cracking reaction of n-butane was performed on the systems formed in Examples 1 to 3 and Comparative Example 1, the conversion rate, the selectivity, and the yield of lower olefins (ethylene and propylene) depending on the reaction were analyzed, and the results are shown in Table 1. The reaction experiment was performed at a pressure of 1 bar and 600 °C, and n-butane at a flow rate of 20 ml/min and a nitrogen gas mixture at a flow rate of 10 ml/min were injected as a feed.

TABLE 1

| | | | | | Comparative |
|---|---|---|---|---|---|
| | | Example 1 | Example 2 | Example 3 | Example 1 |
| Conversion rate (%) | | 64.1 | 69.8 | 76.9 | 54.3 |
| Selectivity | Methane | 9.6 | 7.0 | 5.8 | 13.1 |
| (%) | Propylene | 24.7 | 24.3 | 25.5 | 21.4 |
| | C4═ | 11.5 | 12.5 | 14.1 | 7.3 |

| | | | | |
|---|---|---|---|---|
| Yield (%) | 24.2 | 24.5 | 25.3 | 22.5 |

Referring to Table 1, when a dehydrogenation-cracking reaction is performed by mixing ZSM-5 (Si/Al=140) as a cracking catalyst and a cobalt-platinum/boron oxide-alumina catalyst as a dehydrogenation catalyst according to Examples 1 to 3, it was confirmed that the conversion rate was increased and the yield of lower olefins (ethylene and propylene) was increased, as compared with the case of using only a cracking catalyst according to Comparative Example 1. In addition, it was confirmed that the selectivity for olefins was increased and simultaneously the selectivity for methane was decreased. In particular, when the dehydrogenation catalyst and the cracking catalyst were included at a weight ratio of 4:1 according to Example 3, it was confirmed that the conversion rate and the yield were the highest, and the selectivity for methane was the lowest.

### <Experimental Example 3> Fluidized Bed Dehydrogenation-Cracking Catalyst Reaction of n-Butane

The cracking reaction of n-butane was performed on the system formed in Example 2, the conversion rate, the selectivity, and the yield of lower olefins (ethylene and propylene) depending on a temperature change were analyzed, and the results are shown in Table 2.

TABLE 2

| Reaction temperature | | 580 °C | 600 °C | 620 °C |
|---|---|---|---|---|
| Conversion rate (%) | | 62.6 | 69.8 | 72.7 |
| Selectivity | Methane | 6.5 | 7.0 | 9.7 |
| (%) | Propylene | 24.4 | 24.3 | 26.2 |
| | C4═ | 14.6 | 12.5 | 11.3 |

| | | | |
|---|---|---|---|
| Yield (%) | 20.2 | 24.5 | 28.8 |

Referring to Table 2, it was confirmed that the conversion rate and the yield of olefins were increased, but the selectivity for methane was also increased as the reaction temperature was increased. Therefore, it was confirmed that a reaction temperature of 580 to 600 °C where both an increased yield of olefins from saturated hydrocarbons and minimized methane production were satisfied is the optimal reaction temperature.

### <Experimental Example 4> Fluidized Bed Dehydrogenation-Cracking Catalyst Reaction of Propane

The cracking reaction of propane was performed on the systems formed in Example 2 and Comparative Example 1, and the conversion rate, the selectivity, and the yield of lower olefins (ethylene and propylene) depending on the cracking reaction are shown in Table 3. The reaction experiment was performed at a pressure of 1 bar and 600 °C, and propane at a flow rate of 10 ml/min and a nitrogen gas mixture at a flow rate of 20 ml/min were injected as a feed. The total weight of the catalysts according to Example 2 and Comparative Example 1 was adjusted to 0.4 g.

TABLE 3

| | | | Comparative |
|---|---|---|---|
| | | Example 2 | Example 1 |
| Conversion rate (%) | | 45.1 | 14.5 |
| Selectivity | Methane | 3.9 | 21.6 |
| (%) | Propylene | 64.1 | 31.3 |
| | C4═ | 4.7 | 2.3 |

| | | |
|---|---|---|
| Yield (%) | 31.7 | 10.3 |

Referring to Table 3, when a dehydrogenation-cracking reaction is performed by mixing ZSM-5 (Si/Al=140) as a cracking catalyst and a cobalt-platinum/boron oxide-alumina catalyst as a dehydrogenation catalyst according to Example 2, it was confirmed that the conversion rate was increased and the yield of lower olefins (ethylene and propylene) was increased, as compared with the case of using only a cracking catalyst according to Comparative Example 1. In addition, it was confirmed that the selectivity for olefins was increased and simultaneously the selectivity for methane was decreased.

Although the exemplary embodiments of the present invention have been described above, the present invention is not limited to the exemplary embodiments but may be made in various forms different from each other, and those skilled in the art will understand that the present invention may be implemented in other specific forms without departing from the spirit or essential feature of the present invention. Therefore, it should be understood that the exemplary embodiments described above are not restrictive, but illustrative in all aspects.

1. A method for producing olefins from saturated hydrocarbons, the method comprising: bringing a saturated hydrocarbon feedstock into contact with a mixture of a dehydrogenation catalyst and a cracking catalyst in a circulating fluidized bed reactor.

2. The method for producing olefins from saturated hydrocarbons of claim 1, wherein the saturated hydrocarbon is a C3 to C10 saturated hydrocarbon.

3. The method for producing olefins from saturated hydrocarbons of claim 1, wherein a weight ratio between the dehydrogenation catalyst and a cracking catalyst is 2:1 to 5:1.

4. The method for producing olefins from saturated hydrocarbons of claim 1, wherein the bringing of a saturated hydrocarbon feedstock into contact with a mixture of a dehydrogenation catalyst and a cracking catalyst is performed at 300 to 800 °C.

5. The method for producing olefins from saturated hydrocarbons of claim 1, wherein a dehydrogenation reaction with the dehydrogenation catalyst and an olefin decomposition reaction with the cracking catalyst are performed in a single reactor.

6. The method for producing olefins from saturated hydrocarbons of claim 1, wherein the dehydrogenation catalyst is a metal active component including cobalt and platinum supported on an alumina carrier including boron.

7. The method for producing olefins from saturated hydrocarbons of claim 6, wherein the boron is supported at 0.5 to 2 wt % with respect to the weight of the alumina carrier.

8. The method for producing olefins from saturated hydrocarbons of claim 6, wherein the cobalt is supported at 1 to 5 wt % with respect to the weight of the alumina carrier.

9. The method for producing olefins from saturated hydrocarbons of claim 6, wherein the platinum is supported at 0.001 to 0.05 wt % with respect to the weight of the alumina carrier.

10. The method for producing olefins from saturated hydrocarbons of claim 1, wherein the cracking catalyst is selected from the group consisting of a SAPO molecular sieve, a ZSM molecular sieve, an MCM molecular sieve, and a combination thereof.

11. The method for producing olefins from saturated hydrocarbons of claim 1, wherein the olefin is propylene and butene.

12. The olefins produced by the method for producing olefins from saturated hydrocarbons of claim 1.
